(19) United States
(12) Patent Application Publication
(10) Pub. No.: US 2009/0221898 A1
(43) Pub. Date: Sep. 3, 2009

(54) Shaped surgical tool

(76) Inventors: W. Daniel Hillis, Encino, CA (US); Leroy E. Hood, Seattle, WA (US); Roderick A. Hyde, Redmond, WA (US); Eric C. Leuthardt, St. Louis, MO (US); Nathan P. Myhrvold, Medina, WA (US); Clarence T. Tegreene, Bellevue, WA (US)

Correspondence Address: SEARETE LLC;CLARENCE T. TEGREENE, 1756 - 114TH AVE., S.E., SUITE 110, BELLEVUE, WA, 98004, US

(21) Appl. No.: 12/074,257

(22) Filed: Feb. 28, 2008

(51) Int. Cl. A61B 5/05; A61B 19/00; A61B 17/00

(52) U.S. Cl. 600/407; 128/898; 606/1

(57) ABSTRACT

A custom medical device may be fabricated based on a patient image with a rapid prototyping machine.

### SUMMARY

In one embodiment a method of producing a customized surgical tool comprises obtaining image data corresponding to a patient body region, processing the image data to produce fabrication data, and rapid prototyping the customized surgical tool according to the fabrication data.

In another embodiment a shaped surgical tool comprises a self following, substantially rigid structure of a material suitable for insertion in living tissue of a user, the self following, substantially rigid structure having a shape defined by a user-specific route corresponding to a risk-defined routing through the living tissue.

In another embodiment a system comprises an imaging system operative to provide a data set representative of a region of a patient, path optimization circuitry operative to receive the data set representative of a region of a patient and responsive to the data set representative of a region of a patient to define a self-following path, and a rapid prototyping machine responsive to the defined self-following path to produce an insertable device configured to follow the self-following path.

In another embodiment a method comprises providing image data corresponding to a patient body region to produce fabrication data, receiving a customized surgical tool shaped according to the fabrication data, utilizing the customized surgical tool in contact with the patient body region, and removing the customized surgical tool from contact with the patient body region.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 shows a system comprising an imaging system, path optimization circuitry, and a rapid prototyping machine.

FIG. 2 shows a spiral-shaped insertable device.

FIG. 3 shows a system comprising an imaging system, path optimization circuitry, and a rapid prototyping machine.

FIG. 4 shows a shaped surgical tool.

FIG. 5 shows a shaped surgical tool.

FIG. 6 is a flow chart depicting a method.

FIGS. 7-10 depict variants of the flow chart of FIG. 6.

FIG. 11 is a flow chart depicting a method.

FIGS. 12-14 depict variants of the flow chart of FIG. 11.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Certain medical applications call for a tool created especially for the application. For example, a brain surgeon may wish to reach a target area of the brain with a needle while avoiding certain areas of the brain. In such an application, one may image the brain, determine the area(s) to be reached and the area(s) to be avoided and create a shape that achieves this, and rapid prototype an instrument having this shape. Following are related embodiments.

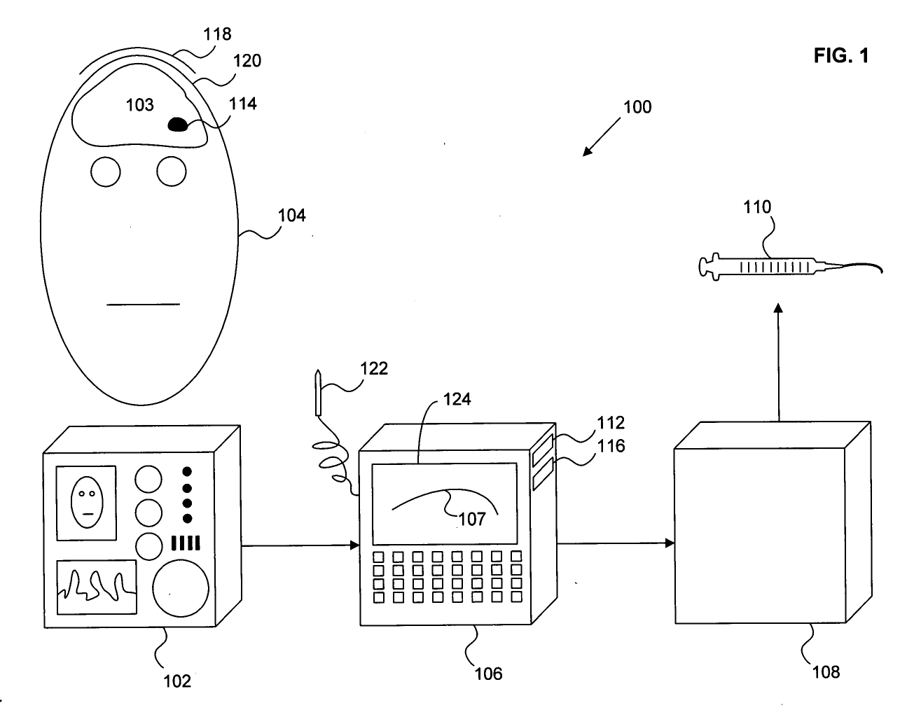

In a first embodiment, shown in FIG. 1, a system 100 comprises an imaging system 102 operative to provide a data set representative of a region 103 of a patient 104, path optimization circuitry 106 operative to receive the data set representative of a region 103 of a patient and responsive to the data set representative of a region 103 of a patient to define a self-following path 107, and a rapid prototyping machine 108 responsive to the defined self-following path 107 to produce an insertable device 110 (such as a biopsy needle, a scalpel, or a different kind of tool) configured to follow the self-following path 107.

Rapid prototyping technology is known to those skilled in the art and many technologies may be implemented as the rapid prototyping machine 108, for example, Stereolithography, Fused Deposition Modeling, and/or Electron Beam Melting. The rapid prototyping machine 108 may include one or more of a range of other processes that can make customized shapes on demand, including: subtractive processes, such as CNC machining, laser-cutting, waterjet cutting, electric-discharge machining; casting using a 3-D-printed master or mold; and/or forming processes, such as computer-controlled bending of metal tubing. The rapid prototyping machine 108 may, for example, be configured to fabricate a mandrel (not shown) that may include a depression in the shape of the desired self-following path 107, where the insertable device 110 may be shaped by using the mandrel as a guide. Further, one skilled in the art may combine one or more techniques, including but not limited to those mentioned above, in the rapid prototyping machine 108. Although the rapid prototyping machine 108 is shown in FIG. 1 as a single machine, it may in some embodiments include any number of different machines, which may be on a scale much larger or smaller than is shown in FIG. 1.

The insertable device 110 may include a metal such as surgical steel or titanium, a plastic such as polypropylene or polycarbonate, glass, a different material, or a combination of several different materials.

The imaging system 102 may include, but is not limited to, an MRI system, a PET system, a CT system, an ultrasound system, an x-ray system, or a different type of imaging system.

The path optimization circuitry 106 operative to receive the data set representative of a region 103 of a patient 104 and responsive to the data set representative of a region 103 of a patient 104 to define a self-following path 107 may further include: avoidance logic 112 configured to define at least one region 114 of prohibited travel of the insertable device 110; alignment structure logic 116 configured to provide data representative of an alignment tool 118 complementary to the insertable device 110, which may assist in inserting the insertable device 110 along a planned trajectory, and which may further be configured to provide conforming data representative of a surface substantially conforming to an outer surface 120 of the patient 104, which may include data representative of a surface substantially conforming to a patient cranial region, where in FIG. 1 the outer surface 120 is that of a patient cranial region.

The system may further include a user input device 122 coupled to the path optimization circuitry 106, wherein the path optimization circuitry 106 is responsive to user interaction with the user input device 122. For example, as shown in FIG. 1, the user input device 122 is a writing instrument configured to write on a screen 124 such that the path optimization circuitry 106 may receive information related to the writing on the screen. The screen 124 may be configured to display image data received from the imaging system 102, alone or along with data marking regions such as sensitive regions that should not be traversed by an instrument, such that the user may draw the desired path according to the display on the screen 124. The screen 124 may further be configured to display an overlay corresponding to image data, which may include identifiers such as the location of the brain and/or sensitive regions, where the overlay may include shading and/or colors to show the identifiers. The screen 124 may further be configured to display image data from different angles, allowing the user to rotate the image display. Further, where the path optimization circuitry 106 is configured to evaluate different areas of the image according to their sensitivity to the passage of a surgical tool, the path optimization circuitry 106 may be configured to calculate a score corresponding to a selected path and display this score on the screen 124 such that a user may optimize the score. Although the user input device 122 is shown and described above as a writing instrument, in other embodiments the user input device 122 may have a different form, such as a device configured to receive a user selection of an assortment of instruments.

Although the path optimization circuitry 106 is shown symbolically as a computer, the path optimization circuitry 106 may take a different form. For example, the path optimization circuitry 106 may be integral to the imaging system 102. Or, the path optimization circuitry 106 may be housed in a simple device that does not receive user input. There are many forms that the path optimization circuitry 106 may take and one skilled in the art may readily adapt the path optimization circuitry 106 to fit a chosen setup.

The avoidance logic 112 and the alignment structure logic 116 are also shown symbolically as a component of a computer. However, as described above with reference to the path optimization circuitry, the avoidance logic 112 and/or the alignment structure logic 116 may take a different form. Further, the path optimization circuitry 106 may include other components not described. For example, the path optimization circuitry 106 may include circuitry for selecting paths through preferred areas rather than avoiding non-preferred areas. Or, the path optimization circuitry 106 may be configured to rank areas based on their accessibility and select a route based on an algorithm that optimizes a path for to minimize damage to a patient.

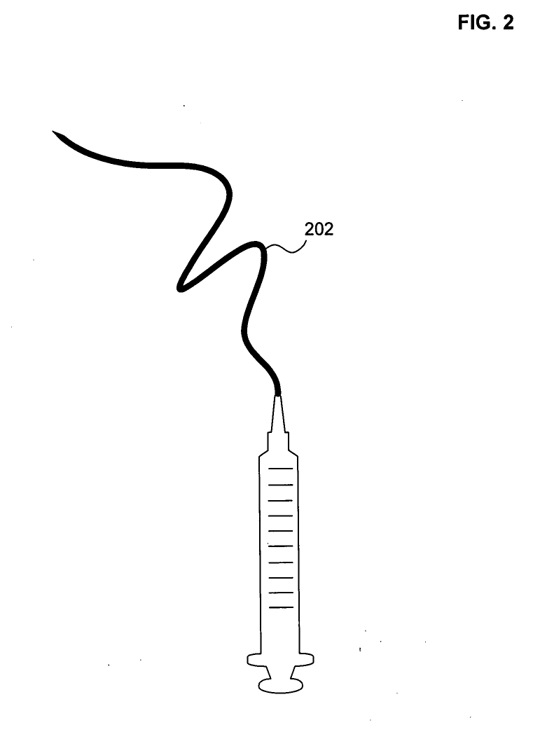

The insertable device 110 that is configured to follow a self-following path such as the self-following path 107 shown in FIG. 1 may be a spiral 202 such as that shown in FIG. 2, an arc such as that of the insertable device 110 shown in FIG. 1, or a different shape. The arc and the spiral are just two examples of different shapes that the insertable device 110 may take, including but not limited to regular, irregular, two-dimensional and/or three-dimensional shapes.

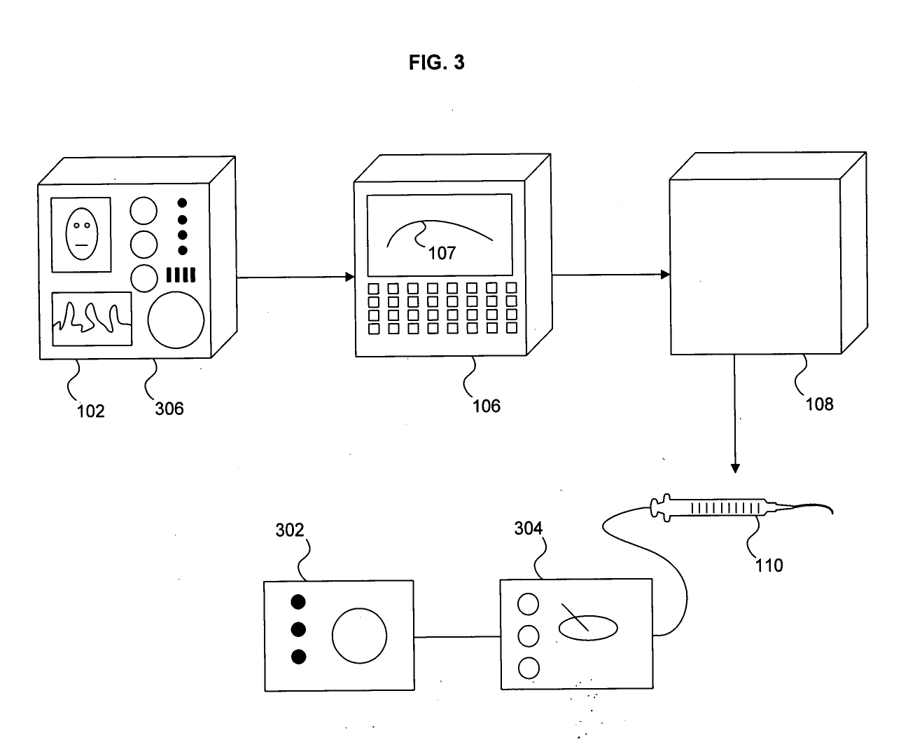

The system may further include an energy exchange system 302 (shown in FIG. 3) arranged to exchange energy with the insertable device. The energy exchange system 302 may be, for example, a system for exchanging heat with the insertable device 110 where the insertable device 110 includes a shape memory alloy (i.e., increasing or decreasing the temperature of the insertable device 110). The energy exchange system 302 may, in the case where the insertable device 110 includes a shape memory alloy, be configured to change the shape of the insertable device. For example, the energy exchange system 302 may be configured to exchange energy with the insertable device 110 in order to bend or elongate the insertable devices 110. Or, the energy exchange system 302 may be configured to heat all or a portion of the insertable device 110 for cauterization or for other reasons.

The system may further include a system 304 arranged to control the insertable device 110. For example, a steerable, insertable device is described in U.S. Pat. No. 6,551,302 entitled STEERABLE CATHETER WITH TIP ALIGNMENT AND SURFACE CONTACT DETECTOR to Rosinko et al., which is incorporated herein by reference. The system 304 may be configured to control the shape, the position, or some other parameter of the insertable device 110. For example, an insertable device 110 may include a guide wire (not shown), where applying mechanical force to the guide wire may move the insertable device 110. Or, the insertable device 110 may include a shape memory alloy as described previously with respect to the energy exchange system 302, where in this case exchanging energy between the shape memory alloy and the energy exchange system 302 is configured to adjust the insertable device 110 in order to steer or otherwise control the insertable device 110. There are many ways of steering and/or adjusting an insertable element and one skilled in the art may incorporate other ways not described to control the insertable device 110.

The system may further include a system 306 for imaging the insertable device 110 when it is inserted into the patient. As shown in FIG. 3, the imaging system 306 for imaging the insertable device 110 is the same as the system 102 that is operative to provide a data set representative of a region 103 of a patient 104, however in other embodiments they may be completely different systems, or they may be substantially different systems that share some components. Further, the system 306 for imaging the insertable device 110 may include components incorporated in and/or on the insertable device 110 for imaging within the patient and/or for locating the insertable device 110 within the patient, and/or it may include components not previously mentioned.

The system may further include a sterilizer, not shown, configured to disable a biomaterial proximate to the insertable device 110. The sterilizer may be configured to deliver heat and/or ultraviolet radiation to the insertable device 110, and/or it may be configured to pass a fluid configured to disable a biomaterial proximate to at least a portion of the insertable device 110. There are many technologies for sterilizing and one skilled in the art may substitute other sterilizing technologies for those previously mentioned.

Although the patient region 103 being imaged in FIG. 1 is a head, it may in other embodiments be a different part of the body, and/or the body may not be a human body but an animal including domestic, marine, research, zoo, farm animals, fowl and sports animals, or pet animals, such as dogs, cats, cattle, horses, sheep, pigs, goats, rabbits, chicken, birds, fish, amphibian and reptile. Although the insertable device 110 is shown as a needle, it need not be a needle and may include, for example, a scalpel, clamp, or a different type of surgical tool.

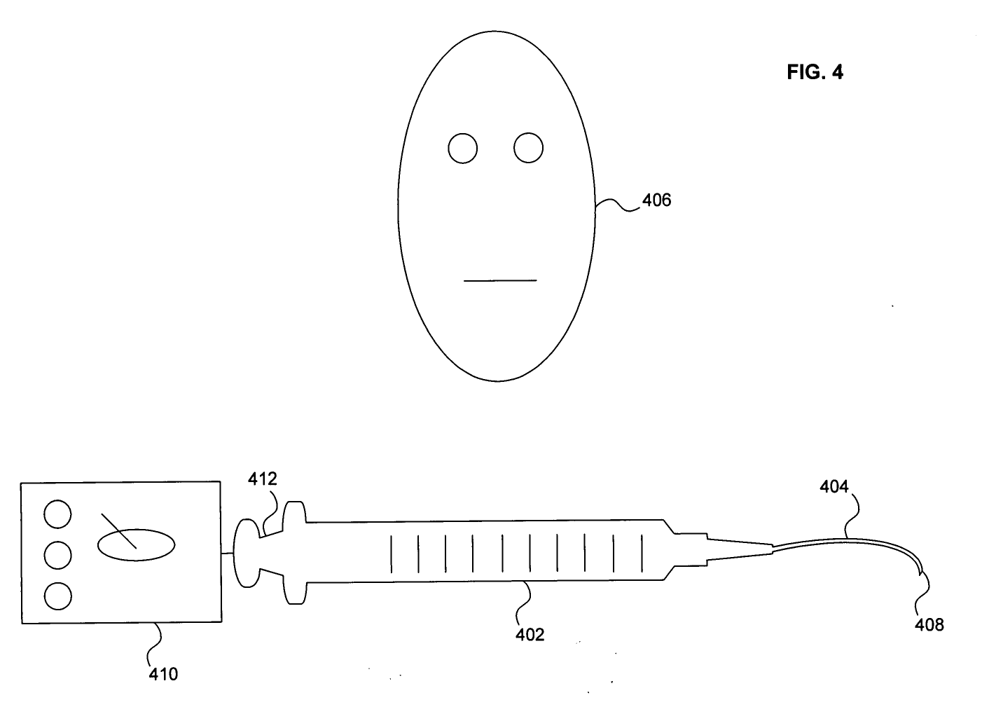

In one embodiment shown in FIG. 4, a shaped surgical tool 402 (in this embodiment, the shaped surgical tool 402 is a biopsy needle) comprises a self following, substantially rigid structure 404 of a material suitable for insertion in living tissue of a user 406, the self following, substantially rigid structure 404 having a shape defined by a user-specific route corresponding to a risk-defined routing through the living tissue. In this embodiment, the self following, substantially rigid structure 404 is arc-shaped, similar to the shape of the insertable device 110 shown in FIGS. 1 and 3, however the shape may include a hook, an arc, a spiral (such as the spiral shown in FIG. 2), or a different self following shape.

The shape is defined by a user-specific route corresponding to a risk-defined routing through the living tissue of a user 406. For example, as described with respect to FIG. 1, the “user-specific route” may be determined by imaging a patient region and using path optimization circuitry 106 in order to define a “risk-defined routing through the living tissue of a user 406”. However there may be other ways of determining a “user-specific route corresponding to a risk-defined routing.” For example, a practitioner may identify a target area to reach with the shaped surgical tool 402 and may, based on general anatomical knowledge, wish to avoid a region proximate to the target area and decide on a shape for a shaped surgical tool 402 based on this knowledge, and create or obtain a shaped surgical tool 402 having this specific shape.

The shape may be dynamically variable, in some cases in response to a user input. For example, as described with respect to FIG. 3, an energy exchange system 302 and/or system 304 may be arranged to move, direct, change the shape of, or otherwise change the shaped surgical tool 402, in the case where the shaped surgical tool 402 includes a shape memory alloy or a different mechanism for changing shape. The shape of the shaped surgical tool 402 may be substantially two dimensional, as in the arc-shaped insertable device 110 shown in FIG. 1, or it may be substantially three-dimensional, as in the spiral 202 shown in FIG. 2. Further, the shape need not be a regular shape and may be irregular. The shaped surgical tool 402 may include a control structure 410 at an end opposite the insertion end. For example, in U.S. Pat. No. 5,769,086 entitled CONTROL SYSTEM AND METHOD FOR AUTOMATED BIOPSY DEVICE to Ritchart et al., which is incorporated herein by reference, the shaped surgical tool 402 includes a control structure arranged move, rotate, and position the shaped surgical tool 402. This is one example of how a controller may be incorporated to control a shaped surgical tool 402 or other insertable device. Other examples include, but are not limited to, a controller configured to bend a shaped surgical tool 402 and/or a controller that is not automated but is user-controlled.

In one embodiment the shaped surgical tool 402 may further include a portion 412 suitable for grasping by a practitioner. The portion 412 suitable for grasping by a practitioner need not be shaped as the exemplary embodiment in FIG. 4 shows, but may be proportionally larger or smaller than shown as compared with the self following, substantially rigid structure 404, and may be more or less irregularly shaped than is shown in FIG. 4.

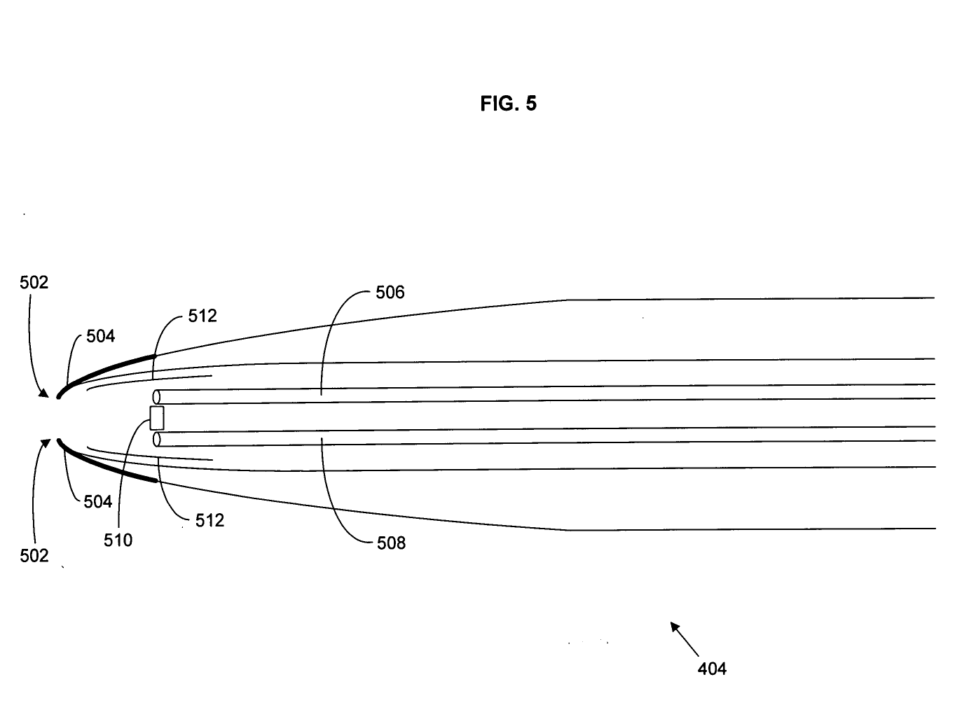

The shaped surgical tool 402 may include a sampling structure 502, as shown in FIG. 5, at an insertion end 408, where the sampling structure 502 shown is a simple device that operates similarly to a tweezer. The sampling structure 502 shown in FIG. 5 is just one example of such, and those skilled in the art may be familiar with other structures. For example, in U.S. Pat. No. 2,496,111 entitled BIOPSY NEEDLE to Henry Turkel, which is incorporated herein by reference, the biopsy needle includes a cutting needle. Other sampling structures may be incorporated in a device depending on the type of device, the function of the sampling structure, and/or depending on other considerations.

The shaped surgical tool 402 may further include a cauterizer 504. For example, in U.S. Pat. No. 5,578,030 entitled BIOPSY NEEDLE WITH CAUTERIZATION FEATURE to John M. Levin, which is incorporated herein by reference, the biopsy needle includes a cauterization feature to cauterize the wound caused by the taking of a tissue specimen and the tissues in contact with the biopsy needle. The cauterizer 504 may be, for example, an electrically conductive region arranged to receive electrical energy and convert it to heat at the insertion end 408 of the self-following, substantially rigid structure 404.

The shaped surgical tool 402 may include a first biofluid guiding conduit 506 at least partially within the self following, substantially rigid structure 404. The first biofluid guiding conduit 506 may be arranged to deliver a biofluid to the user 406 and/or to receive a biofluid from the user 406, where a biofluid may include blood, pharmaceuticals, or a different type of biofluid. The shaped surgical tool 402 may further include a second biofluid guiding conduit 508 different from the first biofluid guiding conduit 506 and at least partially within the self following, substantially rigid structure 404, wherein the second biofluid guiding conduit 508 is arranged to deliver or receive a biofluid from the user 406. Although two biofluid guiding conduits 506 and 508 are shown, other embodiments may have a different number of biofluid guiding conduits. Further, although FIG. 5 is shown with one biofluid guiding conduit 506 to deliver a biofluid to the user 406, in a different embodiment all biofluid guiding conduits may be arranged to receive a biofluid from a user, or a biofluid guiding conduit may be arranged to deliver a biofluid to a user under some circumstances and to receive a biofluid from a user under other circumstances. There are many different ways of configuring a biofluid guiding conduit 506 and/or 508 within a shaped surgical tool 402 and one skilled in the art may configure them according to the particular design of the instrument.

The shaped surgical tool 402 may further include an imaging device 510 proximate to the self following, substantially rigid structure 404. The imaging device 510 may be located at an insertion end 408 of the self-following, substantially rigid structure 404. Or, the imaging device may be at a different location. For example, the imaging device 510 may be located at an insertion end 408 of the self-following, substantially rigid structure to image the tissue that the shaped surgical tool 402 is cutting through. Or, an array of imaging devices 510 may be included on the self-following, substantially rigid structure to image substantially all of the tissue surrounding the self-following, substantially rigid structure 404. Other applications may call for different configurations of imaging devices 510 and one skilled in the art may configure imaging devices 510 according to the design.

In one embodiment the self following, substantially rigid structure 404 may include an extendable core 512 of a material suitable for insertion in living tissue of a user. The extendable core 512 may include a shape memory alloy and/or the extendable core 512 may have a shape that is dynamically variable. The extendable core 512 may, in some embodiments, be an extension of the shaped surgical tool 402 that is smaller than the shaped surgical tool 402 and may be extended in order to reach areas unreachable with the shaped surgical tool 402. Or, the extendable core 512 may include devices for cutting that are only exposed when the shaped surgical tool 402 reaches the area for cutting. These are just a few examples of the ways in which an extendable core 512 may be used with respect to a shaped surgical tool 402.

In another embodiment, the self-following, substantially rigid structure 404 may act as a guide path for placement of electrodes or other neuromodulating constructs (such as light source, heating and/or cooling element, etc.), for delivery of drug and/or molecular therapies, for placement of an acoustic or ultrasonic source, for placement of an optical fiber, or for placement of a different device or material, particularly in regions in the brain that may be difficult to access through straight trajectories from the surface of the head or brain. Example of such locations include the mesial temporal lobe and associated structures such as the hippocampus, the insula, and regions of the hypothalamus. A spiral or other non-linearly shaped structure 404 could allow placement of stimulating electrodes or other neuromodulating devices in these regions to treat medical diagnoses such as epilepsy, psychiatric disorders, or behavior disorders such as over eating/obesity.

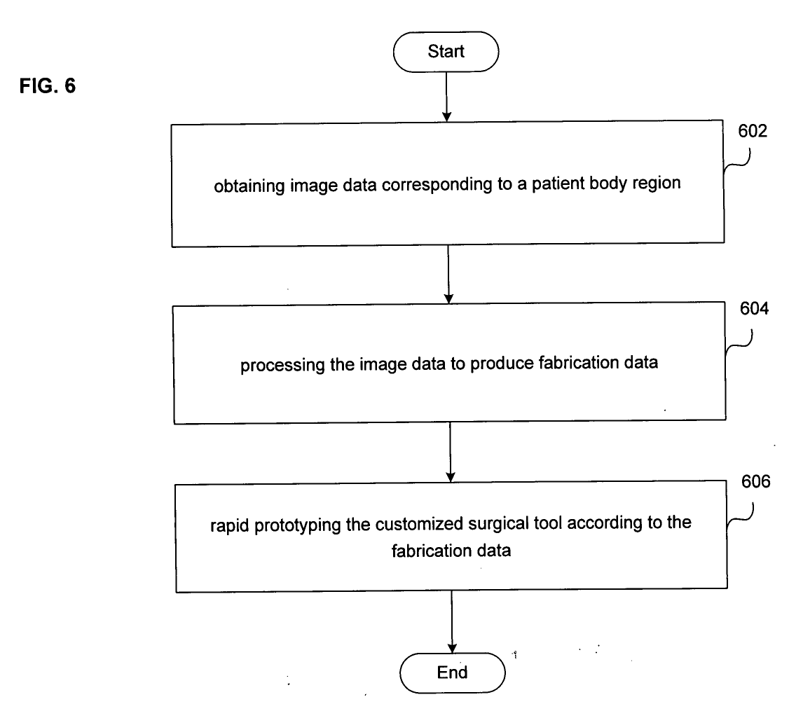

In one embodiment, a method of producing a customized surgical tool, shown in the flow chart of FIG. 6, comprises (602) obtaining image data (such as with the imaging system 102 shown in FIG. 1, through data retrieved from a memory, or from another appropriate source) corresponding to a patient body region (such as the region 103 shown in FIG. 1), (604) processing the image data to produce fabrication data (such as with the path optimization circuitry 106 shown in FIG. 1), and (606) rapid prototyping the customized surgical tool according to the fabrication data (for example, with the rapid prototyping machine 108, also shown in FIG. 1).

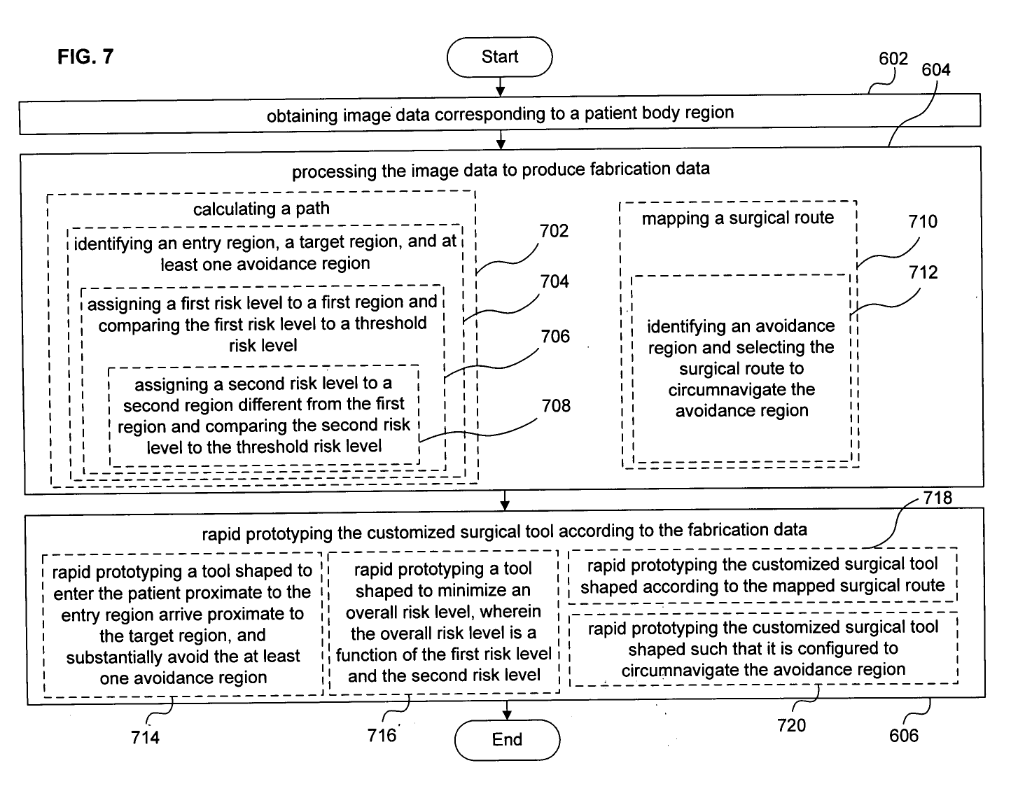

In one embodiment, shown in the flow chart of FIG. 7, (604) processing the image data to produce fabrication data may include (702) calculating a path, which may further include (704) identifying an entry region, a target region, and at least one avoidance region (such as the region of prohibited travel 114 shown in FIG. 1), which may further include (706) assigning a first risk level to a first region and comparing the first risk level to a threshold risk level, which may further include (708) assigning a second risk level to a second region different from the first region and comparing the second risk level to the threshold risk level. (606) Rapid prototyping the customized surgical tool according to the fabrication data may further include (714) rapid prototyping a tool shaped to enter the patient proximate to the entry region, arrive proximate to the target region, and substantially avoid the at least one avoidance region. (606) Rapid prototyping the customized surgical tool according to the fabrication data may further include (716) rapid prototyping a tool shaped to minimize an overall risk level, wherein the overall risk level is a function of the first risk level and the second risk level.

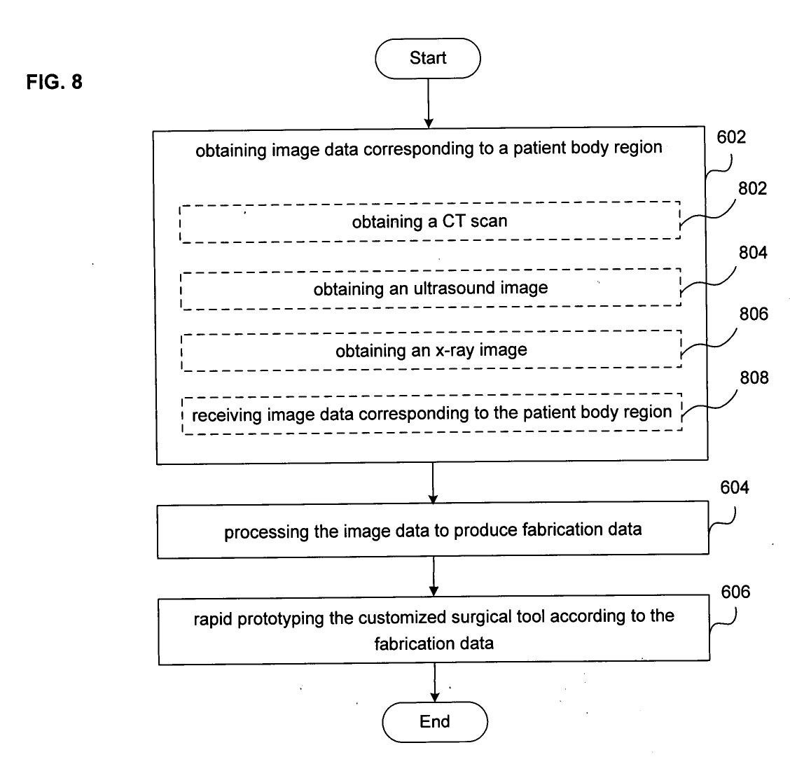

In another embodiment, also shown in FIG. 7, (604) processing the image data to produce fabrication data may include (710) mapping a surgical route, which may further include (712) identifying an avoidance region (such as the region of prohibited travel 114 shown in FIG. 1) and selecting the surgical route to circumnavigate the avoidance region. (606) Rapid prototyping the customized surgical tool according to the fabrication data may further include (718) rapid prototyping the customized surgical tool shaped according to the mapped surgical route, and/or (720) rapid prototyping the customized surgical tool shaped such that it is configured to circumnavigate the avoidance region. Different rapid prototyping technologies have been previously described with respect to the rapid prototyping machine 108 shown in FIG. 1. In different embodiments, shown in the flow chart of FIG. 8, (602) obtaining image data corresponding to a patient body region may include: (802) obtaining a CT scan, (804) obtaining an ultrasound image, (806) obtaining an x-ray image, and/or (808) receiving image data corresponding to the patient body region.

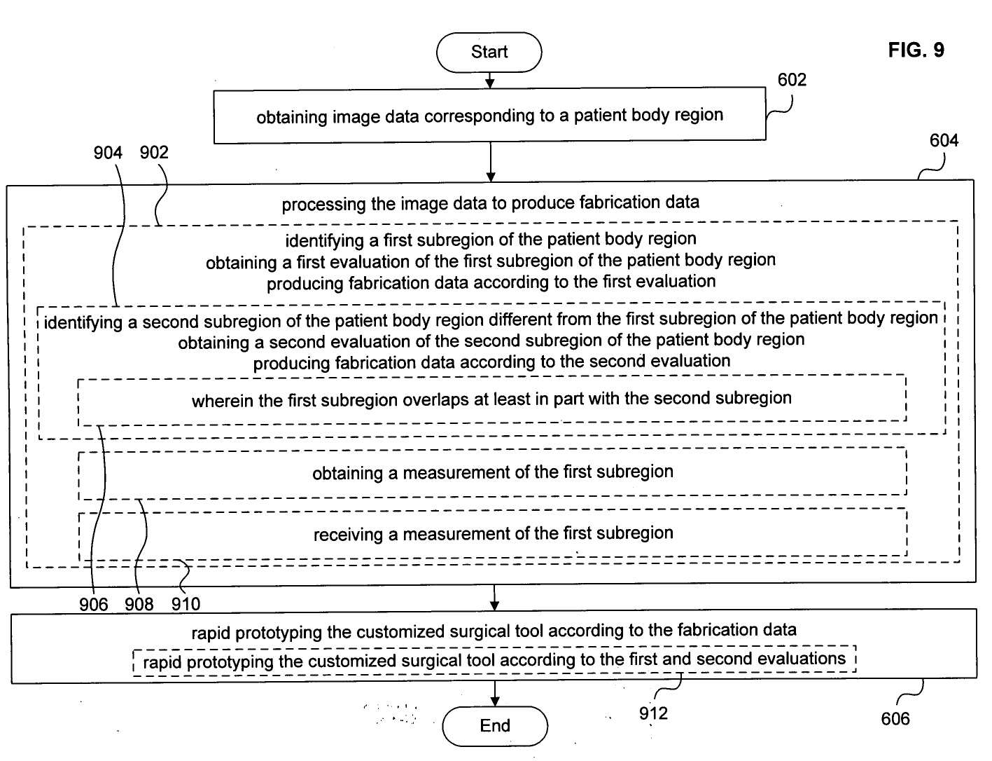

In one embodiment, shown in the flow chart of FIG. 9, (604) processing the image data to produce fabrication data may include (902) identifying a first subregion of the patient body region, obtaining a first evaluation of the first subregion of the patient body region, and producing fabrication data according to the first evaluation, which may further include, (904) identifying a second subregion of the patient body region different from the first subregion of the patient body region, obtaining a second evaluation of the second subregion of the patient body region, and producing fabrication data according to the second evaluation, wherein (906) the first subregion may overlap at least in part with the second subregion. (902) Identifying a first subregion of the patient body region, obtaining a first evaluation of the first subregion of the patient body region, and producing fabrication data according to the first evaluation may further include (908) obtaining a measurement of the first subregion and/or (910) receiving a measurement of the first subregion. (606) Rapid prototyping the customized surgical tool according to the fabrication data may further include (912) rapid prototyping the customized surgical tool according to the first and second evaluations.

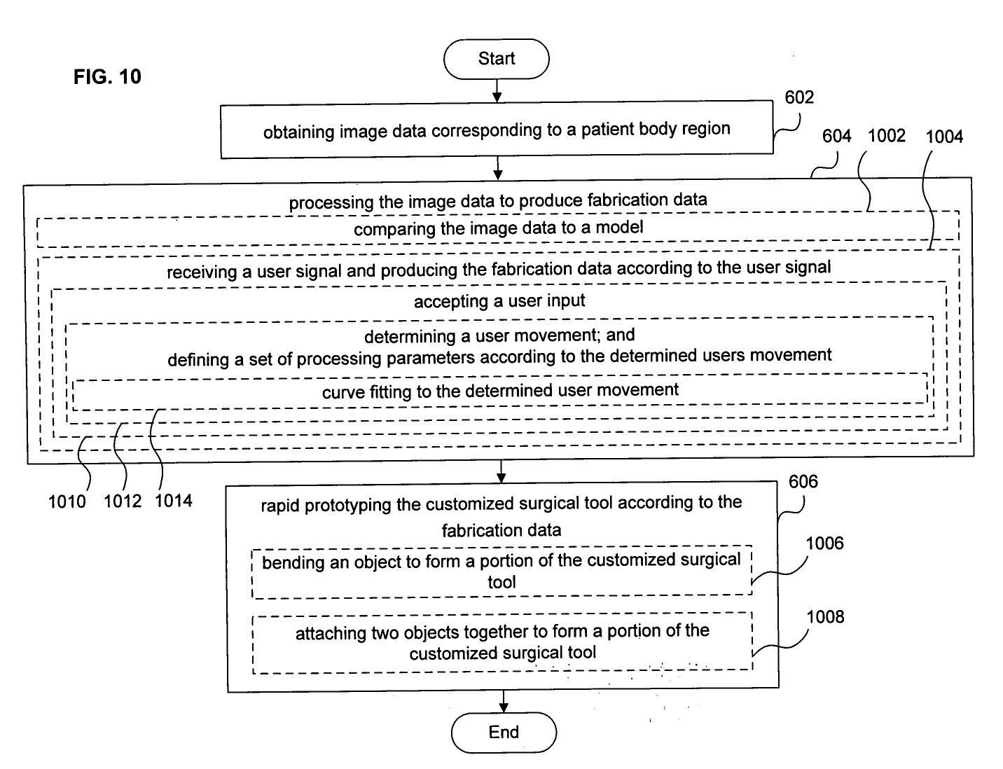

In embodiments shown in the flow chart of FIG. 10, (604) processing the image data to produce fabrication data may further include (1002) comparing the image data to a model (for example, a map including regions of prohibited travel) and/or (1004) receiving a user signal and producing the fabrication data according to the user signal (for example, a user may input a desired shape by selecting from a predetermined array, by drawing a shape that is recognizable by software such as with the user input device 122 as described with respect to FIG. 1, and/or in another way). (1004) Receiving a user signal and producing the fabrication data according to the user signal may further include (1010) accepting a user input, which may further include (1012) determining a user movement and defining a set of processing parameters according to the determined users movement, which may further include (1014) curve fitting to the determined user movement. Further, (606) rapid prototyping the customized surgical tool according to the fabrication data may further include (1006) bending an object to form a portion of the customized surgical tool and/or (1008) attaching two objects together to form a portion of the customized surgical tool.

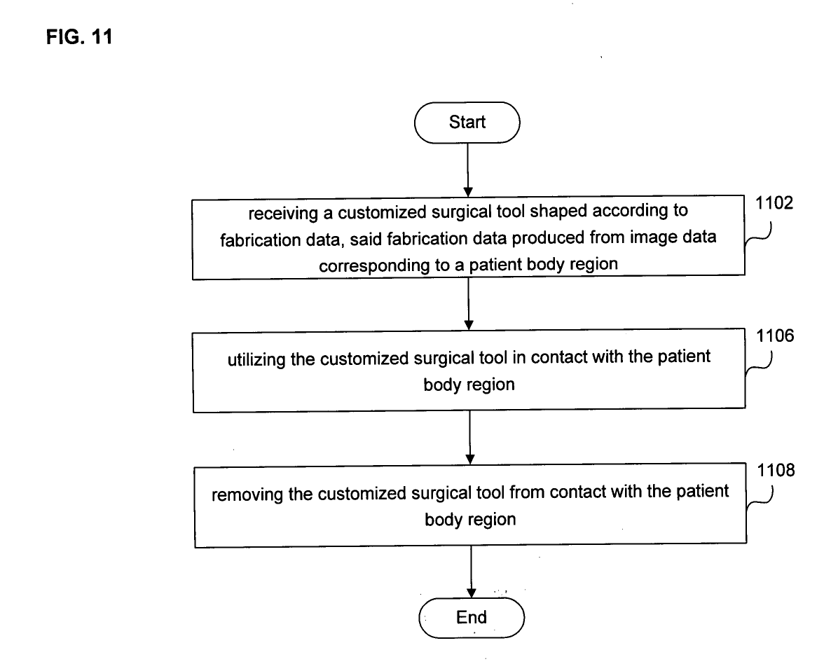

In one embodiment, a method, shown in the flow chart of FIG. 11, comprises (1102) receiving a customized surgical tool shaped according to fabrication data, said fabrication data produced from image data corresponding to a patient body region, (1106) utilizing the customized surgical tool in contact with the patient body region, and (1108) removing the customized surgical tool from contact with the patient body region.

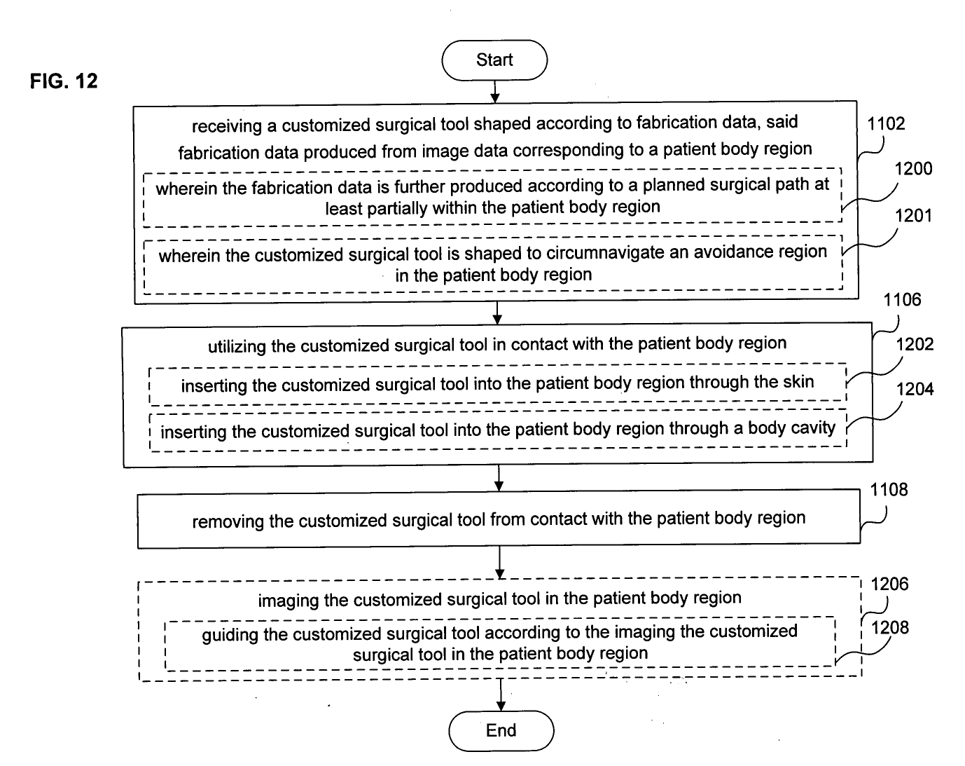

In different embodiments, shown in the flow chart of FIG. 12, (1200) the fabrication data may be produced according to a planned surgical path at least partially within the patient body region, and/or (1201) the customized surgical tool may be shaped to circumnavigate an avoidance region in the patient body region. Further, (1106) utilizing the customized surgical tool in contact with the patient body region may include (1202) inserting the customized surgical tool into the patient body region through the skin and/or (1204) inserting the customized surgical tool into the patient body region through a body cavity. The method may further include, (1206) imaging the customized surgical tool in the patient body region, which may further include (1208) guiding the customized surgical tool according to the imaging the customized surgical tool in the patient body region.

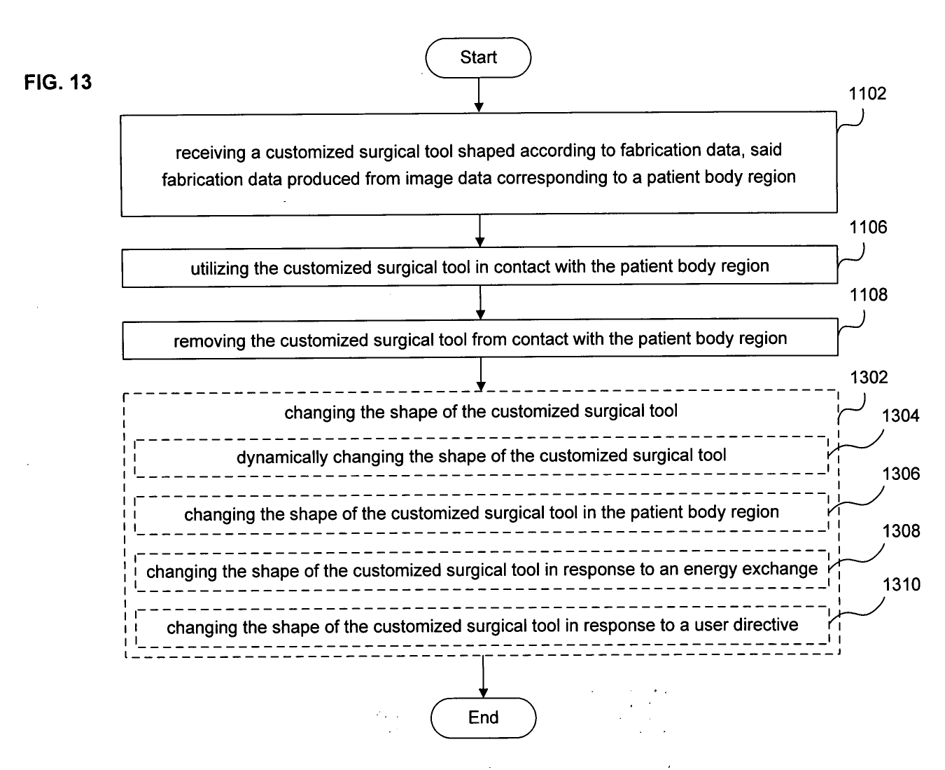

The method may further include, as shown in the flow chart of FIG. 13, (1302) changing the shape of the customized surgical tool, which may further include: (1304) dynamically changing the shape of the customized surgical tool (such as in the case where the customized surgical tool includes a shape memory alloy, where the tool may be configured to bend, telescope, or otherwise change shape in response to a user input), (1306) changing the shape of the customized surgical tool in the patient body region, (1308) changing the shape of the customized surgical tool in response to an energy exchange, and/or (1310) changing the shape of the customized surgical tool in response to a user directive.

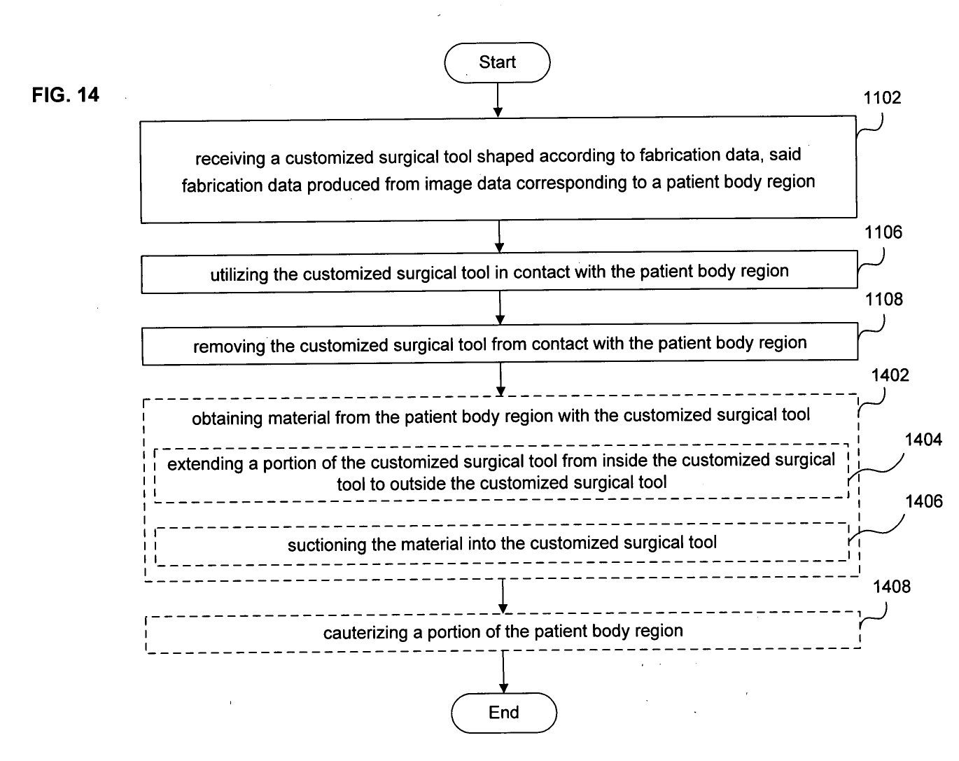

The method may further include, as shown in the flow chart of FIG. 14, (1402) obtaining material from the patient body region with the customized surgical tool, which may further include (1404) extending a portion of the customized surgical tool from inside the customized surgical tool to outside the customized surgical tool and/or (1406) suctioning the material into the customized surgical tool. The method may further include (1408) cauterizing a portion of the patient body region.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various embodiments described herein can be implemented, individually and/or collectively, by various types of electromechanical systems having a wide range of electrical components such as hardware, software, firmware, or virtually any combination thereof; and a wide range of components that may impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs. Those skilled in the art will also appreciate that examples of electromechanical systems include but are not limited to a variety of consumer electronics systems, as well as other systems such as motorized transport systems, factory automation systems, security systems, and communication/computing systems. Those skilled in the art will recognize that electro-mechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices and/or processes in the fashion set forth herein, and thereafter use engineering practices to integrate such described devices and/or processes into image processing systems. That is, at least a portion of the devices and/or processes described herein can be integrated into an image processing system via a reasonable amount of experimentation. Those having skill in the art will recognize that a typical image processing system generally includes one or more of a system unit housing, a video display device, a memory such as volatile and non-volatile memory, processors such as microprocessors and digital signal processors, computational entities such as operating systems, drivers, and applications programs, one or more interaction devices, such as a touch pad or screen, control systems including feedback loops and control motors (e.g., feedback for sensing lens position and/or velocity; control motors for moving/distorting lenses to give desired focuses. A typical image processing system may be implemented utilizing any suitable commercially available components, such as those typically found in digital still systems and/or digital motion systems.

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

Those skilled in the art will recognize that it is common within the art to implement devices and/or processes and/or systems in the fashion(s) set forth herein, and thereafter use engineering and/or business practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation.

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

Those skilled in the art will appreciate that ‘user’ may be representative of a human user, or in some cases a robotic user (e.g., computational entity), and/or substantially any combination thereof (e.g., a user may be assisted by one or more robotic agents). In addition, user, as set forth herein, may in fact be composed of two or more entities. Those skilled in the art will appreciate that, in general, the same may be said of “sender” and/or other entity-oriented terms as such terms are used herein.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

In some instances, one or more components may be referred to herein as “configured to.” Those skilled in the art will recognize that “configured to” can generally encompass active-state components and/or inactive-state components and/or standby-state components, etc. unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

With respect to the appended claims, those skilled in the art will appreciate that recited operations therein may generally be performed in any order. Examples of such alternate orderings may include overlapping, interleaved, interrupted, reordered, incremental, preparatory, supplemental, simultaneous, reverse, or other variant orderings, unless context dictates otherwise. With respect to context, even terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of producing a customized surgical tool, comprising:
obtaining image data corresponding to a patient body region;
processing the image data to produce fabrication data; and
rapid prototyping the customized surgical tool according to the fabrication data.

2. The method of claim 1 wherein processing the image data to produce fabrication data includes:
calculating a path.

3. The method of claim 2 wherein calculating a path further includes:
identifying an entry region, a target region, and at least one avoidance region.

4. The method of claim 3 wherein rapid prototyping the customized surgical tool according to the fabrication data further includes:
rapid prototyping a tool shaped to enter the patient proximate to the entry region, arrive proximate to the target region, and substantially avoid the at least one avoidance region.

5. The method of claim 3 wherein identifying at least one avoidance region further includes:
assigning a first risk level to a first region and comparing the first risk level to a threshold risk level.

6. The method of claim 5 wherein identifying at least one avoidance region further includes:
assigning a second risk level to a second region different from the first region and comparing the second risk level to the threshold risk level.

7. The method of claim 6 wherein rapid prototyping the customized surgical tool according to the fabrication data further includes:
rapid prototyping a tool shaped to minimize an overall risk level, wherein the overall risk level is a function of the first risk level and the second risk level.

8. The method of claim 1 wherein processing the image data to produce fabrication data further includes:
mapping a surgical route.

9. The method of claim 8 wherein rapid prototyping the customized surgical tool according to the fabrication data further includes:
rapid prototyping the customized surgical tool shaped according to the mapped surgical route.

10. The method of claim 8 wherein mapping a surgical route further includes:
identifying an avoidance region and selecting the surgical route to circumnavigate the avoidance region.

11. The method of claim 10 wherein rapid prototyping the customized surgical tool according to the fabrication data further includes:
rapid prototyping the customized surgical tool such that it is configured to circumnavigate the avoidance region.

12. (canceled)

13. (canceled)

14. (canceled)

15. (canceled)

16. (canceled)

17. (canceled)

18. (canceled)

19. The method of claim 1 wherein processing the image data to produce fabrication data further includes:
identifying a first subregion of the patient body region;
obtaining a first evaluation of the first subregion of the patient body region; and
producing fabrication data according to the first evaluation.

20. The method of claim 19 wherein processing the image data to produce fabrication data further includes:
identifying a second subregion of the patient body region different from the first subregion of the patient body region;
obtaining a second evaluation of the second subregion of the patient body region; and
producing fabrication data according to the second evaluation.

21. The method of claim 20 wherein the first subregion overlaps at least in part with the second subregion.

22. The method of claim 20 wherein rapid prototyping the customized surgical tool according to the fabrication data further includes:
rapid prototyping the customized surgical tool according to the first and second evaluations.

23. (canceled)

24. (canceled)

25. (canceled)

26. (canceled)

27. (canceled)

28. (canceled)

29. The method of claim 1 wherein rapid prototyping the customized surgical tool according to the fabrication data further includes:
bending an object to form a portion of the customized surgical tool.

30. The method of claim 1 wherein rapid prototyping the customized surgical tool according to the fabrication data further includes:
attaching two objects together to form a portion of the customized surgical tool.

31. The method of claim 1 wherein rapid prototyping the customized surgical tool according to the fabrication data further includes:
forming a mold; and
conforming a material to the mold.

32. A shaped surgical tool, comprising:
a self following, substantially rigid structure of a material suitable for insertion in living tissue of a user, the self following, substantially rigid structure having a shape defined by a user-specific route corresponding to a risk-defined routing through the living tissue, said shape being dynamically variable in response to a user input.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. A system comprising:
an imaging system operative to provide a data set representative of a region of a patient;
path optimization circuitry operative to receive the data set representative of a region of a patient and responsive to the data set representative of a region of a patient to define a self-following path; and
a rapid prototyping machine responsive to the defined self-following path to produce an insertable device configured to follow the self-following path.

56. (canceled)

57. The system of claim 55 further including an energy exchange system arranged to exchange energy with the insertable device.

58. (canceled)

59. (canceled)

60. (canceled)

61. (canceled)

62. (canceled)

63. The system of claim 55 further including a system arranged to control the insertable device.

64. The system of claim 63 wherein the system arranged to control the insertable device is arranged to control the shape of the insertable device.

65. The system of claim 63 wherein the system arranged to control the insertable device is arranged to control the position of the insertable device.

66. The system of claim 55 further including an imaging system operative to image the insertable device in a region of a patient.

67. (canceled)

68. (canceled)

69. The system of claim 55 wherein the path optimization circuitry operative to receive the data set representative of a region of a patient and responsive to the data set representative of a region of a patient to define a self-following path includes:
alignment structure logic configured to provide data representative of an alignment tool complementary to the insertable device.

70. The system of claim 69 wherein the alignment structure logic configured to provide data representative of an alignment tool complementary to the insertable device is further configured to provide conforming data representative of a surface substantially conforming to an outer surface of the patient.

71. The system of claim 70 wherein the conforming data representative of a surface substantially conforming to an outer surface of the patient includes data representative of a surface substantially conforming to a patient cranial region.

72. (canceled)

73. The system of claim 55 wherein the rapid prototyping machine is a metal fabrication machine.

74. (canceled)

75. The system of claim 55 further including a sterilizer configured to disable a biomaterial proximate to the insertable device.

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)
